(19) United States
(12) Patent Application Publication
(10) Pub. No.: US 2004/0014144 A1
(43) Pub. Date: Jan. 22, 2004

(54) Method of screening for substances acting on MSK1

(75) Inventors: Mogens Winkel Madsen, Virum (DK); Lone Stengelshoj Olsen, Glostrup (DK); Manianne Scheel Fjording, Vaerlose (DK)

Correspondence Address: BIRCH STEWART KOLASCH & BIRCH, PO BOX 747, FALLS CHURCH, VA, 22040-0747, US

(73) Assignee: Leo Pharmaceutical Products Ltd. A/S

(21) Appl. No.: 10/452,591

(22) Filed: Jun. 3, 2003

Related U.S. Application Data
(62) Division of application No. 09/673,128, filed on Oct. 11, 2000.
Application No. 09/673,128 is a 371 of international application No. PCT/DK00/00505, filed on Sep. 13, 2000.
(60) Provisional application No. 60/159,092, filed on Oct. 13, 1999.

(51) Int. Cl. G01N 33/53

(52) U.S. Cl. 435/7.1

(57) ABSTRACT

In a method of identifying substances acting as inhibitors of NF-κB activation by MSK1 or MSK2, MSK1 or MSK2 are contacted with a predetermined amount of one or more test substances in the presence of an NF-κB subunit or a complex of NF-κB subunits, and a test substance is identified as an inhibitor of NF-κB activation by determining a decrease in NF-κB activation by MSK1 or MSK2 in the presence of said test substance compared to the level of NF-κB activation in the absence of said test substance.

### FIELD OF THE INVENTION

The present invention relates to a method of screening for substances acting on mitogen- and stress-activated protein kinase-1 and -2 (MSK1 and MSK2) with a view to identifying potential antiinflammatory compounds.

### BACKGROUND OF THE INVENTION

Proinflammatory cytokines, including tumour necrosis factor type α (TNF-α) and interleukin 1 type β (IL-1β), play an important role in initiating the inflammatory response. Acute and chronic inflammation is involved in diseases such as rheumatoid arthritis, osteoarthritis, inflammatory bowel disease and toxic shock syndrome, and inhibition of the production, release or blocking the action of these cytokines may by used as an anti-inflammatory treatment. Several compounds have been developed targeting the production or action of the proinflammatory cytokines. For example, the TNF-α synthesis in monocytes can be inhibited by inhibition of the phosphodiesterase type IV (PDE IV) by rolipram (1,2). In addition, TNF-α and IL-1β production from monocytes can be blocked by inhibition of the p38 MAP kinase by SB203580 (3). Neutralizing antibodies against TNF-α have been used in treatment of rheumatoid arthritis (4). U.S. Pat. No. 5,783,664 describes a cytokine suppressive anti-inflammatory drug binding protein which is the apparent target for a class of pyridinyl imidazoles which appear to arrest expression of IL-1 and TNF at the translation and transcription level.

Recently, two novel protein kinases designated mitogen- and stress-activated protein kinase-1 and -2 (MSK1 and MSK2) have been identified (5). The MSKs can be activated by growth factors such as EGF and TPA, and by stress, such as by UV light, arsenite and H₂O₂. MSK integrates two different cellular pathways, the mitogen and the stress-induced pathways. The MSKs contain two kinase domains like the RSK kinases, and have a high homology with isoforms of RSK. The activation by growth factors can be inhibited by PD98059, a MEK1 inhibitor, and the stress-induced activation of MSK1 can be inhibited by SB203580, a p38 kinase inhibitor. MSK1 is therefore a novel downstream substrate for the ERK and the p38 MAP kinases (5). In addition, the previously identified protein kinase C inhibitor, Ro318220 has shown to be a potent inhibitor of MSK1 activity in vitro (5).

The cloning and expression of MSK1 and MSK2 is disclosed in (8). It is suggested that MSK1 and MSK2 may regulate the transcription of genes coding for COX-2 and IL-1β, both of which are mediators of inflammatory reactions, as well as the induction of the COX-2 protein, and that this regulation is the result of the phosphorylation by MSK1 and MSK2 of CREB and AFT-1, the transcription factors believed to be responsible for control of COX-2 expression. It is therefore suggested in (8) that compounds exerting an antiinflammatory effect by inhibiting COX-2 and IL-1β may be identified in a method of screening for compounds inhibiting the activity of MSK1 or MSK2.

### SUMMARY OF THE INVENTION

The present inventors have shown that when the human monocytic cell line THP-1 or peripheral blood mononuclear cells (PBMC) are treated with Ro318220, the lipopolysaccharide (LPS) induced secretion of TNF-α and IL-1β is strongly inhibited. The same inhibition is observed when these cell types are treated with a combination of PD98059 and SB203580. These findings confirm that MSK1 is downstream of the p38 MAP kinase in the regulation of the cytokine production of TNF-α and IL-1β. The fact that LPS stimulation of monocytes and activation of p38 MAP kinase have also been found to activate the ERK kinase pathway (6), and MSK1 seems to converge the signals from the two MAP kinases, makes MSK1 an ideal molecular target for the discovery of compounds that inhibit the production of proinflammatory cytokines such as TNF-α and IL-1β

Accordingly, the present invention relates to a method of identifying substances acting as inhibitors of the production of proinflammatory cytokines, the method comprising contacting MSK1 with a predetermined amount of one or more test substances, wherein a test substance is identified as an inhibitor of the production of a proinflammatory cytokine when MSK1 activity is decreased in the presence of said substance relative to the activity of MSK1 in the absence of said test substance.

It has been shown (Caivano, M. and Cohen, P.: J. Immunol. 164, 3018-3025 (2000); 8) that MSK2 is activated in a like manner on LPS stimulation of macrophages. MSK2 is therefore likely to act through the same pathways as MSK1 and may be inhibited by similar compounds.

Thus in another aspect, the invention relates to a method of identifying substances acting as inhibitors of the production of proinflammatory cytokines, the method comprising contacting MSK2 with a predetermined amount of one or more test substances, wherein a test substance is identified as an inhibitor of the production of a proinflammatory cytokine when MSK2 activity is decreased in the presence of said substance relative to the activity of MSK2 in the absence of said test substance.

The finding that the production of other proinflammatory cytokines than IL-1β, notably TNF-α, can be affected by the activation or inhibition of MSK1 and MSK2 suggests that an as yet undisclosed pathway through at least one transcription factor other than CREB may be involved. In the course of research leading to the present invention, it has surprisingly been found that MSK1 and MSK2 also play an important role for the activation of the transcription factor NF κB.

Consequently, the invention further relates to a method of identifying substances acting as inhibitors of NF-κB activation by MSK1 or MSK2, the method comprising contacting MSK1 or MSK2 with a predetermined amount of one or more test substances in the presence of NF-κB or a subunit or fusion protein thereof and identifying a test substance as an inhibitor of NF-κB activation by determining a decrease in NF-κB activation by MSK1 or MSK2 in the presence of said test substance compared to the level of NF-κB activation in the absence of said test substance.

It may also be desirable to test compounds or agents for any activation effect which they may have on MSK1. It will be appreciated that the knowledge of such properties is useful not only for the development of new useful therapies and the like, but also in order to screen compounds and agents of interest for undesirable side effects, such as might be expected to occur with activation of production of proinflammatory cytokines such as TNF-α and/or IL-1β.

Thus, in a further aspect, the invention relates to a method of identifying substances activating MSK1 or MSK2, the method comprising contacting MSK1 or MSK2 with a predetermined amount of one or more test substances, wherein a test substance is identified as an activator or MSK1 or MSK2 when MSK1 or MSK2 activity is increased in the presence of said substance relative to the activity of MSK1 or MSK2 in the absence of said test substance.

### DETAILED DESCRIPTION OF THE INVENTION

Compounds, agents and substances to be tested in the methods of the present invention include synthetic and naturally-derived isolated or pure chemical compounds, as well as biological mixtures and compositions and the like which may or may not be fully characterized. Any synthetic or natural compound may be tested according to the invention. The screening method of the invention is particularly suitable for testing synthetic small organic molecules, particularly organic heterocyclic compounds, for example imidazoles, thiazoles, indolylcarbazoles, pyrolopyrimidines, quinazolines, oxindoles, pyridopyrimidine, pyridopyrimidone, amino benzophenones and flavones. Such test substances may either be tested individually or as a combination of two or more of the substances.

It will be appreciated that tested substances need not be completely purified, although isolation and partial purification will be necessary in the case of naturally derived substances in order to perform the screening with any accuracy.

In general terms, compounds and the like which are deemed suitable for such screening are tested by contacting them with MSK1 or MSK2 in an assay medium under suitable conditions to determine whether MSK1 or MSK2 activity is affected by the presence of the compound. Thus, the assay is performed in the presence and absence (control) of the test compound to be screened in an amount which is determined to be suitable, considering factors which will be known to those of skill in the art, such as the physical and chemical properties of the compound. It will be clear that the amount of compound employed can also be varied, should the need arise, and that the reaction conditions can be varied according to the convenience and objectives of the individual performing the assay, within the limits of the general knowledge of the art, and routine experimentation. Thus, the presence, absence and amount of various reagents, the time and temperature of the reaction, and the like can be adjusted according to the needs of a particular situation.

More specifically, the present screening method may be designed as a cell-based assay in which cells expressing MSK1 or MSK2 are contacted with the test substance, and any effect on the expression of proinflammatory cytokines by the cells is determined (cf. for instance Example 1 herein). To avoid any possible interference with other cellular signaling factors or pathways in the initial screening stage, it is, however, preferred that the MSK1 or MSK2 is employed in substantially pure form in the assay, i.e. purified from cellular proteins or other cellular components.

In the present screening method, MSK1 or MSK2 may be contacted with the test substance or substances in the presence of a suitable substrate which may comprise CREB or ATF-1, or fusion protein thereof. In the present context, the term “fusion protein” is intended to indicate the fusion of CREB or ATF-1 with another protein or peptide such as glutathione S-transferase which is provided to facilitate the purification of the substrate during substrate production. Activated MSK1 or MSK2 act by phosphorylating the substrate, e.g. CREB, which in turn binds to a binding site for the transcription factor in the gene coding for a protein such as a cytokine, typically in the promoter part thereof, thus initiating the transcription of said protein. In the assay, phosphorylation of the substrate may be determined, e.g. by adding a radioactive isotope of phosphorus such as ³²P or ³³P which is then incorporated in the substrate on phosphorylation by MSK1 or MSK2 and may be measured by determining the radioactivity incorporated in the substrate (cf. Example 8 herein). Inhibition of phosphorylation of the substrate by a test substance may be determined as the reduction of incorporated radioactivity relative to a control sample with no test substance added.

Alternatively, phosphorylation of the substrate may be determined using an antibody which is reactive with the substrate in its phosphorylated form only. Such antibodies are commercially available from different chemical suppliers, e.g. New England Biolabs. Antibodies bound to the phosphorylated substrate may then be detected in a number of ways known to the person skilled in the art, e.g. by in-plate binding assay or radiometric assays. The in-plate binding assays include enzyme-catalyzed colorimetric and luminescent read-outs and time-resolved fluorescence (e.g. europium cryptate (EuK) from Packard Instrument Company). The radiometric assays using ³²P or ³³P or SPA (scintillation proximity assay, from Amersham International).

Alternatively, a synthetic substrate capable of being phosphorylated by MSK1 or MSK2 may be used in the present methods, e.g. a peptide with the amino acid sequence Glu-Ile-Leu-Ser-Arg-Arg-Pro-Ser-Tyr-Arg-Lys, Gly-Arg-Pro-Arg-Thr-Ser-Ser-Phe-Ala-Glu-Gly, Lys-Lys-Arg-Asn-Arg-Thr-Leu-Ser-Val-Ala, Lys-Lys-Arg-Asn-Lys-Thr-Leu-Ser-Val-Ala or Lys-Lys-Leu-Asn-Arg-Thr-Leu-Ser-Val-Ala. MSK1 and MSK2 are capable of phosphorylating these substrates at a serine residue in the peptide sequence.

In general, when subjected to a screening method of the invention, a compound is considered to be a potential inhibitor of the production of proinflammatory cytokines such as TNF-α and IL-1β and a potential anti-inflammatory agent if a tested concentration results in at least a 10% inhibition of the MSK1 assay. Practically speaking, levels of inhibition of at least 25%, more preferably at least 50% or greater, are preferred. Persons of skill in the art will appreciate that evaluation of the ultimate medical or pharmaceutical utility of compounds discovered by the methods of the invention will depend on further factors such as potency, selectivity, specificity, cost, solubility and toxicity, among others. However, the invention provides a very useful means of determining potential candidates for such further testing. Such potential candidates are preferably selective inhibitors of MSK1 or MSK2, i.e. they have an IC₅₀ value which is about 100 times or more lower for MSK1 or MSK2 than for other kinases.

The proinflammatory cytokines may suitably be selected from the group consisting of TNFα, IL-1β, IL-6 and IL-8. The cytokines TNF-α, IL-1β and IL-6 have been shown to be very important in the inflammatory process. More specifically, TNF-α seems to be important for initiating and amplifying the inflammatory process. Blocking the action of TNF-α in patients with rheumatoid arthritis with either neutralizing antibodies or by soluble recombinant TNF receptor has led to significant clinical improvements (Elliott M J et al. Arthritis & Rheumatism 36, 1681-1690 (1993); Moreland L W et al. New England J. Med. 337, 141-147 (1997)). The importance of IL-1 in inflammation was shown in the collagen induced arthritis in a mouse model where neutralizing antibodies against IL-αβ prevented cartilage and bone destruction (Joosten L A B et al.: J. Immunol. 163, 5049-5055 (1999)). In the same mouse model, development of arthritis was prevented by blocking antibodies against the IL-6 receptor (Takagi N. et al. Arthritis & Rheumatism 41, 2117-2121 (1998). IL-8 seems to play a role in psoriasis (Br. J. Dermatol. 138, 63-70 (1998).

For use in a screening method of the invention, the MSK1 or MSK2 proteins may suitably be prepared by recombinant DNA techniques. Thus, a DNA sequence encoding MSK1 or MSK2 may suitably be isolated substantially as disclosed in Example 3 herein, or as disclosed in (5) or (8).

Briefly, a DNA sequence encoding MSK1 or MSK2 may suitably be obtained by isolating total RNA from cells producing MSK1 or MSK2, such as monocytes, and subjecting the RNA to reverse transcription followed by PCR amplification substantially as disclosed in (5) using suitable oligonucleotide primers based on the published DNA sequences of human MSK1 (GenBank accession no. AF074393), murine MSK2 (GenBank accession no. AF074714) and (8).

The DNA encoding MSK1 or MSK2 isolated in this manner may then be inserted into a suitable expression vector which, dependent on the host cell of choice, may be an autonomously replicating vector, e.g. a plasmid, or be integrated into the genome of the host cell and be replicated together with the chromosome into which it has been integrated. In the expression vector, the DNA coding for MSK1 or MSK2 may be operably linked to additional segments required for transcription of the DNA, such as a promoter and sequences upstream of the promoter. The term “operably linked” indicates that the segments are arranged so that they function in concert for their intended purpose, e.g. transcription initiates in the promoter and proceeds through the DNA sequence coding for MSK1 or MSK2. The promoter may be any DNA sequence which shows transcriptional activity in the host cell of choice and may be derived from genes encoding proteins which are either homologous or heterologous to the host cell. However, when the host cell is a mammalian cell, the promoter is preferably a CMV promotor, SV40 early promoter or a TK promoter.

The DNA sequence encoding MSK1 or MSK2 may also, if necessary, be operably linked to a suitable terminator. The expression vector may further comprise elements such as polyadenylation signals, transcription enhancer sequences and translation enhancer sequences.

The procedures used to ligate the DNA sequence coding for MSK1 or MSK2 with the promoter sequences and optionally other sequences and to insert them into suitable expression vectors are well known to persons skilled in the art, cf. Sambrook et al., Molecular Cloning: A Laboratory Manual, 2^{nd} Ed., Cold Spring Harbor Laboratory, Cold Spring Harbor, N.Y., 1989.

The host cell into which the DNA sequence encoding MSK1 or MSK2 is introduced may be any cell which is capable of producing MSK1 or MSK2 and includes bacteria, yeast and higher eukaryotic cells such as insect and mammalian cells.

Examples of bacterial host cells which, on cultivation, are capable of producing MSK1 or MSK2 are grampositive bacteria such as strains of Bacillus or gramnegative bacteria such as Escherichia coli. The transformation of the bacteria may be effected by protoplast transformation or by using competent cells in a manner known per se (cf. Sambrook et al., supra). When MSK1 or MSK2 is produced in bacteria such as E. coli, the protein may be produced as a fusion protein, which facilitates protein purifcation. As an example MSK1 or MSK2 can e subcloned in a vector which expresses the MSK1 or MSK2 as a fusion protein to the GST (Gluthathione S-Transferase). The expression of the GST fusion protein is under control of the lac repressor (the product of the lad gene). The lac repressor binds to the promoter of the GST fusion protein and represses its expression. Upon addition of IPTG (isopropyl-β-D-thiogalactoside), the promoter of the fusion protein is released from its repression, and the GST fusion protein is expressed. Proteins fused to GST can be purified from lysed cells because of the high affinity of GST for Glutathione immobilised on Sepharose beads.

Examples of suitable yeast cells include strains of Saccharomyces such as strains of Saccharomyces cerevisiae or Saccharomyces kluyveri. Method of transforming yeast cells with heterologous DNA and producing heterologous proteins therein are described in, e.g. U.S. Pat. No. 4,599,311, US 4,931,373, US 4,870,008, US 5,037,743 and US 4,845,075. Transformed cells are selected by a phenotype determined by a selectable marker, typically drug resistance or the ability to grow in the absence of a particular nutrient. The DNA sequence encoding MSK1 or MSK2 may be preceded by a signal sequence and optionally a leader sequence, e.g. as described in the above-cited references.

Examples of suitable mammalian cell lines are the COS-1 (ATCC CRL 1650), BHK (ATCC CRL 1632, ATCC CCL 10), CHO-K1 (ATCC CCL 61), 293 (ATCC CRL-1573), THP-1 (ATCC TIB-202), HL-60 (ATCC CCL-240) and the RAW 264.7 (ATCC TIB-71) cell lines. A currently preferred mammalian cell line for producing MSK1 or MSK2 is COS-1 which is an African green monkey fibroblast like cell line. Methods of transfecting mammalian cells and expressing DNA sequences introduced therein are described in, e.g., Kaufman and Sharp, J. Mol. Biol. 159, 1982, pp. 601-621; Southern and Berg, J. Mol. Appl. Genet. 1, 1982, pp. 327-341; Loyter et al., Proc. Natl. Acad. Sci. USA 79, 1982, pp. 422-426; Wigler et al., Cell 14, 1978, p. 725; Corsaro and Pearson, Somatic Cell Genetics 7, 1981, p. 603; Graham and van der Eb, Virology 52, 1973, p. 456; and Neumann et al., EMBO J. 1, 1982, pp. 841-845, Yao, J., Mackman, N., Edgington, T. S. & Fan, S. T. (1997), J. Biol. Chem, 272, 17795-17801.

Transformation of insect cells and production of heterologous proteins therein may be conducted as described in U.S. Pat. No. 4,745,051, US 4,879,236, US 5,155,037, US 5,162,222, EP 397,485. The insect cell line used as the host may suitably be a Lepidoptera cell line, such as Spodoptera frugiperda cells or Trichoplusia ni cells (cf. U.S. Pat. No. 5,077,214). An example of a insect cell line is Sf9 (Invitrogen). Culture conditions may suitably be as described in, e.g. WO 89/01029 or WO 89/01028, or any of of the aforementioned references. The Sf9 cell line can easily be transfected and cloned cDNA's expressed using a baculovirus vector as indicated in the references mentioned above.

The transformed or transfected host cell may then be cultured in a suitable nutrient medium under conditions permitting the production of MSK1 or MSK2.

The medium used to culture the cells may be any conventional medium suitable for growing the host cells such as minimal or complex media containing appropriate supplements, e.g. a variety of factors ensuring overexpression of MSK1 or MSK2 such as LPS, EGF or a phorbol ester, e.g. PMA. Suitable media are available from commercial suppliers or may be prepared according to published recipes (e.g. in the catalogues of the American Type Culture Collection). For use in the present screening method, it is preferred that the resulting MSK1 or MSK2 is subsequently recovered from the culture by conventional procedures involving separating the host cells from the medium by filtration or centrifugation, followed by cell lysis, precipitating the proteinaceous components of the supernatant or filtrate by means of a salt, e.g. ammonium sulphate, and subjected to purification procedures such as ion exchange chromatography, gel filtration chromatography, affinity chromatography or the like.

In a currently favoured embodiment of the present method, MSK1 is contacted with the test substance or substances in the presence of a substrate comprising an NF-κB subunit or a complex of two identical (homodimeric) or different (heterodimeric) subunits thereof.

The transcriptional activator NF-κB is ubiquitously found as an inactive complex in the cytoplasm bound to its inhibitory subunit IκB. It has been found to be an important regulator implicated in the control of expression of TNF-α and IL-1β. Drug discovery efforts targeting TNF-α or IL-1β synthesis by repressing the transcription of these genes could be a valuable approach.

NF-κB is a family of structurally and functionally related proteins involved in the regulation of transcription from a wide variety of genes. The NF-κB family of transcription factors is composed of a p50 and p65 (also called RelA) heterodimer but also other NF-κB subunits such as p52, c-rel and RelB may DNA binding, p65, RelB and c-rel have, in addition, trans-activating activity. which share a structurally homologous N-terminal Rel domain which encodes DNA binding and dimerisation functions. The various NF-κB subunits interact to form homo- or heterodimeric complexes. The way the subunits combine may influence their specificity for DNA. For further information on NF-κB, see (9) and the references cited therein.

It has been recognised for some time that NF-κB regulates the transcription and activation of the genes encoding the proinflammatory cytokines IL-1β(9) and TNF-α (10) in response to the induction of the monocytic cell line THP-1 by LPS or a phorbol ester (9) or by staphylococcal enterotoxin A (10). Further work has been carried out to elucidate the nature of the induction of the TNF-α promoter by LPS (7). In a clinical context, it has been found that the spontaneous production of TNF-α and other proinflammatory cytokines is dependent on NF-κB in rheumatoid synovial tissue (11). It has been established that NF-κB and the p38 MAP kinase are activated through separate and distinct pathways (12), and it has been proposed that a kinase downstream in the p38 MAP kinase and ERK pathways phosphorylates and thereby activates NF-κB (13), but the identity of this putative kinase has not previously been disclosed.

The present inventors have found that this putative kinase may be MSK1 and/or MSK2. Experiments conducted by the inventors have shown that when the human monocytic cell line THP-1 cells are treated with Ro318220 or by SB203580 combined with PD98059, the LPS induced NFκB transcriptional activity is strongly inhibited. These observations suggest that MSK1 and MSK2 are downstream of the p38 MAP kinase and ERK in the activation of NFκB transcriptional activity (for details—see Example 2).

In the screening method of the invention of identifying compounds that are potential inhibitors of NF-κB, a test substance is identified as an inhibitor of NF-κB activation by MSK1 or MSK2 when such activation is inhibited by at least 10%, more preferably by at least 25%, such as by at least 50%, in the presence of the test substance relative to activation in the absence of the test substance. The method may be used to identify specific inhibitors of NF-κB activation, i.e. compounds that preferentially inhibit NF-κB rather than another transcription factor, e.g. AP1 and c-myc, using a reporter gene assay, e.g. as proposed in Example 2. Suitable inhibitors of NF-κB activation act by inhibiting MSK1 or MSK2. It is preferred to use substantially purified MSK1 or MSK2 in the screening assay to avoid interference from other factors or components of the cells producing MSK1 or MSK2.

Compounds identified by the present method to be inhibitors of NF-κB activation are believed to be capable of reducing the level of transcription of proinflammatory cytokines mediated by NF-κB not only in an in vitro system such as a cell-based assay, but also in vivo such as in a mammal suffering from an inflammatory condition.

In a further aspect, the invention therefore relates to a method of of reducing the NF-κB mediated production of proinflammatory cytokines in mammalian cells the method comprising contacting cells, which cells express activated MSK1 or MSK2 resulting in increased activation of NF-κB and production of proinflammatory cytokines, with a sufficient amount of a substance identified by the method indicated above to be an inhibitor of NF-κB activation by MSK1 or MSK2 for a sufficient period of time to effect a reduction in the production of proinflammatory cytokines by said cells.

The cells contacted with the inhibitor of NF-κB activation, whether in vitro or in vivo, are cells naturally producing proinflammatory cytokines such as leukocytes, peripheral blood mononuclear cells, monocytes, T-cells, macrophages, mast cells and endothelial cells. By contacting such cells with sufficient amounts of an inhibitor of NF-κB activation, which may preferentially act by inhibiting MSK1 or MSK2 activity, it may be possible to inhibit excessive production of proinflammatory cytokines. It is therefore envisaged that inhibitors of NF-κB activation may be employed in the prevention or treatment of diseases or disorders characterised by overproduction of proinflammatory cytokines. Examples of such diseases are inflammatory diseases or conditions such as rheumatoid arthritis, osteoarthritis, psoriatic arthritis, enteropathic arthritis, systemic lupus erythematosis, dermatomyositis, polymyositis, sclerodermia, mixed connective tissue disease, Sjogren's disease, systemic sclerosis, amyloidosis, autoimmune hepatitis, ciliary cirrhosis, glomerulonephritis, Graves' disease, diabetes type 1, sepsis, septic shock, endotoxin shock, asthma, adult respiratory distress syndome, chronic pulmonary inflammatory diease, pulmonary sarcoidosis, reperfusion injury, allograft rejection, inflammatory bowel disease such as Crohn's disease and ulcerative colitis, psoriasis, atopic dermatitis, acne and other types of inflammatory dermatitis.

The invention is further described in the following examples which are not in any way intended to limit the scope of the invention as claimed.

### EXAMPLES

Abbreviations

CREB: cyclic AMP-response element binding protein

GST: glutathione S-transferase

IL-1β: interleukin-1β

LPS: lipopolysaccharide

MSK1: mitogen and stress-activated protein kinase-1

NF-κB: nuclear factor-κB

PBMC: peripheral blood mononuclear cells

PKI: peptide inhibitor of cyclic-AMP dependent protein kinase

SDS: sodium dodecyl sulfate

TNF-α: tumor necrosis factor-α

### Example 1

The mononuclear cells (PBMC) were isolated from human peripheral blood by Lymphoprep® fractionation (Nycomed, Norway) and the THP-1 cells were obtained from the American Type Culture Collection (Accession No. TIB-202). Both cell lines were suspended in RPMI 1640 with 2% fetal calf serum (FCS), 2 mM L-glutamine, 100 U/ml penicillin and 100 mg/ml streptomycin. PBMC were preincubated for 30 min. with 1 μM Ro318220, 10 μM PD98059, 1 μM SB204580, or a combination of the compounds, and stimulated for 18 hours with 1 μg/ml LPS (Sigma Chemical Company, St. Louis, Mo., USA). The THP-1 cells were preincubated for 60 min. with 1 μM Ro318220, 1 μM PD98059, 1 μM SB203580, or a combination of the compounds and stimulated for 5 hours with 1 μg/ml LPS. The levels of IL-1β and TNF-α were measured in the culture supernatant by enzyme immuno assays (monoclonal antibodies were obtained from R & D Systems, Abingdon, UK). In PBMC, the secretion of TNF-α and IL-1β were inhibited about 40-60% after SB203580 treatment, 40% after PD98059 and 90% after Ro318220 treatment. Pretreatment with 10 μM PD98059 and 1 μM SB203580 resulted in approximately 90% inhibition. In THP-1 cells, the secretion of TNF-α was inhibited by 30% after 1 μM SB203580 treatment, 5% after 1 μM PD98059 treatment and 50% after 1 μM Ro318220 treatment. Pretreatment with 1 μM PD98059 and 1 μM SB203580 resulted in approximately 60% inhibition.

These findings confirm that MSK1 is downstream of the p38 MAP kinase in the regulation of the cytokine production of TNF-α and IL-1β.

### Example 2

An NFκB-luciferase reporter plasmid (pBIIX) containing two copies of the sequence (5′-ACA GAG GGG ACT TTC CGA GAG-3′ separated by four nucleotides (5′-ATCT-3′) in front of a mouse fos promoter in plasmid pfLUC (a pBluescript-based plasmid with a firefly luciferase encoding sequence) was prepared as disclosed in Saksela, K., & Baltimore, D. (1993), Mol. Cell Biol., 13, 3698-3705. The NFκB binding site from mouse Igκ light chain is underlined (Grilli, M., Chiu, J. J., & Lenardo, M. J. (1993), Int. Rev. Cytol., 143, 1-62).

THP-1 cells were transfected with pBIIX during the log phase of growth using a DEAE-dextran transfection procedure (modified from [11]). Plasmid DNA was prepared and purified using the QIAGEN EndoToxin-free Maxiprep-500 kit (Hilden, Germany). Approximately 3.5×10⁷ cells were resuspended in 1 ml transfection medium (RPMI 1640 supplemented with 2 mM L-glutamine) and incubated with 5 μg of pBIIX, 0.5 μg pRL-TK vector (Renilla luciferase plasmid with a thymidine kinase promoter as a control for transfection efficiency obtained from Promega Inc., Madison, Wis.) plus 500 μg of DEAE-dextran (Sigma). Addition of 10 ml of the transfection medium stopped the transfection. After having been washed with the transfection medium, the cells were resuspended in 7 ml of culture medium. The cells were then distributed into 24-well plates, each well containing 250 μl of cell suspension (approximately 1.25×10⁶ cells) and 1.25 ml culture medium, and they were incubated for 48 h. One hour prior to stimulation with 1 μg/ml LPS (E. coli serotype 055:B5, Sigma), the THP-1 cells were pretreated for 1 hour with 1 μM Ro318220, 1 μM PD98059, 1 μM SB203580, or a combination of the compounds keeping the concentration of the solvent DMSO below 0.2% (v/v). All fluids and dilutions were made with endotoxin free water. Cells were harvested 5 h later, pelleted by centrifugation and washed with 1×PBS before being resuspended in 50 μl of lysis buffer. The cell lysate was assayed for luciferase activity using the Dual luciferase kit as described by the manufacturer (Promega). All transfections were performed in triplicate. Firefly luciferase activity was corrected for transfection efficiency by normalising it to the measured Renilla luciferase activity. Pretreatment of the cells with 1 μM PD98059 had no effect on NFκB transcriptional activity, whereas 1 μM SB203580 or 1 μM Ro318220 resulted in a reduction of the reporter gene activity to 49% and 15%, respectively, of the control value. Moreover, treatment of the cells with 1 μM PD98059 and 1 μM SB203580 in combination resulted a reduction of the luciferase activity to 45% of the control level.

These findings confirm that MSK1 and MSK2 are downstream of the p38 MAP kinase in the regulation of NFκB transcriptional activity.

### Example 3

RT-PCR Cloning of Human MSK1 and Preparation of Activated FLAG-MSK1

FLAG-tagged human MSK1 cDNA was obtained as follows: Total RNA was isolated from THP-1 cells and the MSK1 cDNA was RT-PCR amplified into two overlapping parts using the oligonucleotides 5′-TCCGCTGTCTCCTGGGTTCC-3′ and 5′-GCACTCCTGGCAACATTTGTCACT-3′ (the N-terminal part of MSK1) and the oligonucleotides 5′-GTCAGAGGGGGAGATTCAGGAC-3′ and 5′-ATGAGACCAACGGGAAACATTTTTA-3′ (the C-terminal part of MSK1) as primeres both parts covering an internal Bam HI site. Both PCR products were each ligated into the pCR-Blunt-II TOPO vector (Invitrogen). A FLAG epitope was added to MSK1 by PCR using the N-terminal part of MSK1 cDNA as a template and the oligonucleotides 5′-GAGGTACCGCCACCATGGACTACAAGGACGACGATGACAAGGAGGAGGAG GGTGGCAGCAGCGGCG-3′ (incorporating a KpnI site and the FLAG epitope) and 5′-CCTGAAACAGCTTCTCAGAACTCTG-3′ as primers. This PCR product was then ligated into the pcDNA 3.1(+) vector (Invitrogen) using the Kpn I and the BamH I site. The C-terminal part of MSK1 was excised from the pCR-Blunt-11 TOPO and ligated with pcDNA3.1/FLAG/MSK1(the N-terminal part) using BamH I and EcoR I. The resulting vector was designated pcDNA3.1/FLAG/MSK1.

Cell Culture

COS-1 cells (derived from African green monkey kidney fibroblast-like cell containing wild-type T antigen under control of the SV40 promotor) were obtained from ATCC (ATCC no. CRL-1650) and grown in growth medium (DMEM without phenol red, 10% FCS, 2 mM L-glutamine, 100U penicillin and 100 μg streptomycin/ml) at 37° C. with 5% CO₂. The cells were passaged twice a week by trypsination (0.25% trypsin, 1 mM EDTA in PBS) and were split 1:10. The medium was changed every second or third day. The cell line was regularly tested with the Mycoplasma PCR Primer Set (Stratagene) and found to be free of Mycoplasma. Tissue culture media, FCS, L-Glutamine and penicillin and streptomycin are from Gibco BRL, Gaithersburg, Md., USA.

Transient Transfection of COS-1 Cells

On day one COS-1 cells were seeded in 143 cm² petri dish with a density of 2×10⁴ cells/cm² in growth medium. At day 2 the cells were transfected with 5 μg (total) of pcDNA3.1/FLAG/MSK1 plasmid DNA. COS-1 cells were transfected during the log phase of growth using DOTAP™ (Boehringer-Mannheim, Mannheim, Germany). Plasmid DNA was prepared and purified using the QIAGEN EndoToxin-free Maxiprep-500 kit (Hilden, Germany). Briefly, DNA and DOTAP™ were mixed for exactly 15 min. at 37° C. in the CO₂ incubator. The transfection mixture was hereafter transferred to a 15-ml falcon tube and transfection medium (DMEM with L-Glutamine and Pen./Strep. but without serum) was added to the transfection mixture, followed by addition to the cell monolayer. After 4 hours of incubation with DOTAP™ and plasmids, the medium containing double amount of serum were added to the cells bringing the final concentration of serum up to 10%. The cells were then incubated for 24 hours before cell stimulation with EGF or Anisomycin or TPA.

Immunoprecipitation by Anti-FLAG

Cells were lysed followed by immunoprecipitation using monoclonal anti-FLAG (M2) antibodies to obtain MSK1. The cells were lysed using a lysis-buffer (50 mM HEPES, pH7.5, 150 mM NaCl, 10 mM EDTA, 10 mM Na₄P₂O₇, 100 mM NaF, 1% Triton X-100, 10 μg/ml of Aprotinin (available from Roche) and Leupeptin (available from Roche), 500 μM Pefabloc (available from Roche), 2 mM Na₃VO₄). Immunoprecipitation was carried out at 4° C. with 2 μg anti-FLAG (M2) preadsorbed to 25 μl protein G Sepharose beads in 30 mM HEPES pH7.5, 30 mM NaCl, 0.1% Tween-20. The anti-FLAG M2 monoclonal antibody was obtained from Sigma (cat. no. F-3165). Following the immunoprecipitation the Sepharose beads were washed twice in lysis-buffer and twice in a kinase reaction buffer (25 mM HEPES pH 7.5, 10 mM magnesium acetate, 50 μM ATP).

### Example 4

Preparation of COS-1 Expressed and Activated GST-MSK1

GST-tagged human MSK1 cDNA for eukaryotic expression was obtained as follows: A eukaryotic vector for expression of GST fusion protein was constructed by PCR amplifying the GST and Multi Cloning Site from pGEX-4T-1 (Amersham Pharmacia Biotech) using the oligonucleotides 5′-ACGGCTAGCGATGTCCCCTATACTAGGTTATTGGAAAAT-3′ (incorporating a Nhe I site) and 5′-CAGAGGTTTTCACCGTCATCACC-3′ as primers. The resulting PCR product was then ligated into the pcDNA 3.1 (+) vector using the Nhe I site and an internal Not I site thereby creating a vector designated pcDNA3.1/GST. Human MSK1 cDNA was PCR was amplified from the pcDNA3.1/FLAG/MSK1 using the oligonucleotide 5′-CGAGAATTCGAGGAGGAGGGTGGCAGCAG-3′ (incorporating an EcoR I site) and 5′-GATGCGGCCGCCTAAGCTACTGAGTCCGAGAACTGGA-3′ (incorporating a Not I site) and ligated into the EcoR I and Not I site of the pcDNA3.1/GST vector. The resulting vector was designated pcDNA3.1/GST/MSK1.

Transient Transfection of COS-1 Cells

On day one COS-1 cells were seeded in 143 cm² petri dish with a density of 2×10⁴ cells/cm² in growth medium. At day 2 the cells were transfected with 5 μg (total) of pcDNA3.1/GST/MSK1. COS-1 cells were transfected during the log phase of growth using DOTAP™ (Boehringer-Mannheim, Mannheim, Germany).

Plasmid DNA was prepared and purified using the QIAGEN EndoToxin-free Maxiprep-500 kit (Hilden, Germany). Briefly, DNA and DOTAP™ were mixed for exactly 15 min. at 37° C. in the CO₂ incubator. The transfection mixture was then transferred to a 15 ml falcon tube and transfection medium (DMEM with L-Glutamine and Pen./Strep. but without serum) was added to the transfection mixture, followed by addition to the cell monolayer. After 4 hours of incubation with DOTAP™ and plasmids, the medium containing a double amount of serum was added to the cells bringing the final concentration of serum up to 10%. The cells were then incubated for 24 hours before cell stimulation with EGF or Anisomycin or TPA.

Purification of COS-1 Expressed GST-MSK1

Cells were lysed and the cell lysate are added to a Gluthathione Sepharose 4B column. The GST fusion protein was hereby purified by affinity chromatography using the affinity of the GST molecule to Gluthathione. The GST fusion protein was eluated by adding excess amount of gluthatione, and the eluate was checked by measurement of the absorbance, Coomassie gel staining and Western Blotting using specific antibodies. The purified GST fusion protein was used in a kinase reaction using GST-CREB as substrate in a kinase buffer (25 mM HEPES pH7.5, 10 mM Mg(Ac)₂, 50 μM ATP).

### Example 5

Preparation of E. coli Expressed GST-MSK1

Construction of pGEX/MSK1

GST-tagged human MSK1 cDNA for prokaryotic expression was obtained as follows: Human MSK1 was PCR amplified from the pcDNA/FLAG/MSK1 vector using the oligonucleotides 5′-CGAGAATTCGAGGAGGAGGGTGGCAGCAG-3′ (incorporating an EcoR I site) and 5′-GATGCGGCCGCCTAAGCTACTGAGTCCGAGAACTGGA-3′ (incorporating a Not I site). The PCR product was then ligated into the pGEX-4T-1 vector using the EcoR I-Not I sites. The resulting vector was designated pGEX/MSK1.

Purification of E. coli Expressed GST-MSK1

GST-MSK1 or GST-MSK2 were expressed in E. coli strain BL21. The fusion protein was induced by adding IPTG for 4 hrs, after which the cells were harvested and then lysed by addition of lysozyme. The bacterial lysate was cleared of cellular debris by centrifugation, and the cleared lysate was added to Gluthathione Sepharose 4B column. The fusion protein binds to the matrix, and the bound GST fusion protein was eluated by excess gluthathione. The purified GST fusion protein was used in a kinase reaction using GST-CREB as substrate in a kinase buffer (25 mM HEPES pH7.5, 10 mM Mg(Ac)₂, 50 μM ATP).

### Example 6

GST-CREB Substrate

GST-tagged rat CREB cDNA for E. coli expression was obtained as follows: Total RNA from rat brain was obtained from Clontech and a spliced variant of CREB was RT-PCR amplified using the oligonucleotides 5′-GACTCTGGAGCAGACAACCAGCA-3′ and 5′-ATCCAGTCCATTTTCCACCACATAG-3′ and the PCR product was ligated into the pCR-Blunt-II TOPO vector. The CREB cDNA was excised from the vector using flanking EcoR I sites and ligated into the pGEX-4T-1 vector. The E. coli BL12 strain was transformed with the construct and induced by 1 mM isopropyl-β-D-thiogalactoside for 4 h at 37° C. The GST-CREB fusion protein was then purified on glutathione-Sepharose substantially as described in Example 5.

### Example 7

RT-PCR Cloning of Murine MSK2

Murine MSK1 cDNA was obtained as follows: Total RNA was isolated from the murine macrophage cell line RAW264.7 (ATCC TIB-71) and the MSK2 cDNA was RT-PCR amplified using the oligonucleotides 5′-CGCCATGGGAGACGAGGATGAGGAC-3′ and 5′-GCACCAGGCTCCCGGATCGGA-3′ as primers. The PCR product was then ligated into the pCR-Blunt-II TOPO vector (Invitrogen). The MSK2 cDNA was then subcloned into different expression vectors such as the mammalian expression vector pcDNA3.1(+) (Invitrogen).

### Example 8

Assays Using MSK1

A. FLAG-tagged MSK1 or FLAG-MSK2 cDNA was transfected into the COS-1 cells and stimulated EGF and Anisomycin or TPA. This stimulation was omitted if the propose of the assay was to identify activators of MSK1 and MSK2. Cells were lysed followed by immunoprecipitation using monoclonal anti-FLAG (M2) antibodies and a kinase assay was performed using GST-CREB as a substrate. In brief, cells were lysed in a lysis buffer (50 mM HEPES, pH 7.5, 150 mM NaCl, 10 mM EDTA, 10 mM Na₄P₂O₇, 100 mM NaF, 1% Triton X-100, 10 μg/ml of Aprotinin and Leupeptin, 500 μM Pefabloc, 2 mm Na₃VO₄), followed by immunoprecipitation at 4° C. with 2 μg anti-FLAG (M2) preadsorbed to 25 μl protein G Sepharose beads in 30 mM HEPES pH7.5, 30 mM NaCl, 0.1% Tween-20. Following the immunoprecipitation the Sepharose beads were washed twice in lysis-buffer and twice in a kinase reaction buffer (25 mm HEPES pH 7.5, 10 mM Mg(Ac)₂, 50 μM ATP).

The immunoprecipitated FLAG-MSK1 or FLAG-MSK2 were contacted with the test compound or compounds for 30 min. at 30° C. The kinase reaction was started by adding GST-CREB substrate together with 0.05 μCi γ-³²P-ATP. The reaction was carried out for 20 min at 30° C. with an occasional tapping on the tubes to keep the immunoprecipitation in suspension. The reaction was stopped by adding 2× SDS-sample buffer, boiled and resolved on a SDS-PAGE. The dried SDS-PAGE gel was exposed to a Phospho-Imager screen, and the radioactive GST-CREB bands were quantified by STORM Phospho-Imager (Molecular Dynamics) using ImageQuaNT software.

B. An MSK1 and MSK2 kinase assay using Crosstide as a substrate is performed as follows: The purified GST-MSK1 or GST-MSK2 from either COS-1 cells or E. coli is added to an Eppendorf tube and contacted with the test compound of compounds for 30 min, at 30°. Kinase reaction is started by addition of Crosstide (30 μM), 2.5 μM PKI (peptide inhibitor of cAMP-dependent kinase), 0.1 mM γ-³²P-ATP (100-200 cpm/pmol) in kinase buffer (50 mM Tris-HCl pH 7.5, 0.1 M EGTA, 0.1% mercaptoethanol, 10 mM Mg(Ac)₂. The reaction is terminated after 10 min at 30° C. by pipetting 40 μl assay mixture into a 2×2 cm square of phosphocellulose paper (P81, Whatman, Clifton, N.J.) that binds Crosstide but not ATP, and immersing the paper in a beaker containing 0.5% phosphoric acid. After washing the papers five times with phosphoric acid to remove ATP, followed by one wash in acetone to remove phosphoric acid, the P81 papers are dried, and counted in a scintillation counter, and analyzed for ³²P radioactivity.

C. The purified GST-MSK1 or GST-MSK2 from either COS-1 cells or E. coli was added to an Eppendorf tube and contacted with the test compound of compounds for 30 min, at 30°. Then the kinase reaction was started by adding substrate, e.g GST-CREB or another peptide substrate, together with 0.05 μCi γ-³P-ATP in kinase reaction buffer (25 mM HEPES pH 7.5, 10 mM Mg(Ac)₂, 50 μM ATP). The reaction was carried out for 20 min at 30° C. The reaction was terminated by addition of 2× SDS-sample buffer, boiled and resolved on a SDS-PAGE. The dried SDS-PAGE gel was exposed to a Phospho-Imager screen, and the radioactive GST-CREB bands were quantified by STORM Phospho-Imager (Molecular Dynamics) using ImageQuaNT software.

D. An MSK1 and MSK2 kinase assay for high-throughput screening purposes is performed as follows: The purified GST-CREB is added to a microtiter plate and is allowed to adhere to the plastic coat. Then the purified GST-MSK1 or GST-MSK2 from either COS-1 cells or E. coli are added to the substrate together with kinase buffer (25 mM HEPES pH 7.5, 10 mM Mg(Ac)₂, 50 μM ATP) and the test compound or compounds. The reaction is allowed to proceed for 20 min at 30° C. followed by addition of an anti-phospho-CREB antibody (New England Biolabs). This antibody reacts with CREB phosphorylated at Ser133. A secondary antibody coupled to either HRP (Horseradish Peroxidase) or Europium or another detection molecule is added and the reaction is detected using either enzyme-catalyzed colorimetric or time-resolved fluorescence.

### REFERENCES

1. Griswold, D. E., Webb, E. F., Breton, J., White, J. R., Marshall, P. J., and Torphy, T. J. Effect of selective phosphodiesterase type IV inhibitor, rolipram, on fluid and cellular phases of inflammatory response. Inflammation, 17: 333-344, 1993.

2. Verghese, M. W., McConnell, R. T., Strickland, A. B., Goading, R. C., Stimpson, S. A., Yarnall, D. P., Taylor, J. D., and Furdon, P. J. Differnential regulation of human monocyte-derived TNF-alpha and IL-1beta by type IV cAMP-phosphodiesterase (cAMP-PDE) inhibitors. J. Pharmacol. Exp. Ther. 272: 1313-1320, 1995.

3. Badger, A. M.; Bradbeer, J. N., otta, B., Lee, J. C., Adams, J. L., and Griswold, D. E. Pharmacological profile of SB 203580, a selective inhibitor of cytokine suppressive binding protein/p38 kinase, in animal model og arthritis, bone resorption, endotoxin shock and immune function. J. Pharmacol. Exp. Ther. 279: 1453-1461, 1996.

4. Maini, R. N. and Feldmann, M. Cytokine therapy in rheumatoid arthritis. Lancet, 348: 824-825, 1996.

5. Deak, M., Clifton, A. D., Lucocq, J. M., and Alessi, D. R. Mitogen- and stress-activated protein kinase-1 (MSK1) is directly activated by MAPK and SAPK2/p38, and mediate activation of CREB. EMBO J. 17: 4426-441, 1998.

6. Swantek, J. L., Cobb, M. H., and Geppert, T. D. Jun N-terminal kinase/stress-activated protein kinase (JNK/SAPK) is required for lipopolysaccharide stimulation of tumor necrosis factor alpha (TNF-alpha) translation: Glucocorticoids inhibit TNF-alpha translation by blocking JNK/SAPK. Mol. Cell. Biol. 17: 6274-6282, 1997.

7. Yao, J., Mackman, N., Edgington, T. S. & Fan, S. T. (1997): Lipopolysaccharide induction of the tumor necrosis factor-alpha promoter in human monocytic cells. Regulation by Egr-1, c-Jun, and NF-κB transcription factors. J. Biol. Chem, 272, 17795-17801.

8. WO 99/67283, filed on 08.06.99 and published on 29.12.99.

9. Chen, F., Castranova, V., Shi, X., & Demers, L. M. (1999). New insights into the role of nuclear factor-kB, a ubiquitous transcription factor in the initiation of diseases. Clin Chem, 45, 7-17.

10. N S Trede et al., J. Immunol. 155, 1995, pp. 902-908.

11. J. Bondeson et al., Proc. Natl. Acad. Sci. USA 96, 1999, pp. 5668-5673.

12. S. Wesselborg et al., J. Biol. Chem. 272(19), 1997, pp. 12422-12429.

13. W Vanden Berghe et al., J. Biol. Chem. 273(6), 1998, pp. 3285-3290.

1. A method of identifying substances acting as inhibitors of NF-κB activation by mitogen and stress-activated protein kinase-1 (MSK1), the method comprising
(a) contacting MSK1, said MSK1 being in a form in which it is purified from cellular proteins or other cellular components with a test substance in the presence of a substrate for MSK1,
(b) measuring the level of phosphorylation by MSK1 of said substrate in the presence or absence of said test substance,
(c) identifying a test substance as an inhibitor of MSK1 when MSK1 activity is decreased in the presence of said test substance relative to the MSK1 activity in the absence of said test substance, and
(d) contacting said test substance with cells that express activated MSK1 resulting in increased activation of NF-κB and production of proinflammatory cytokines and identifying a test substance identified as an inhibitor of MSK1 in step (c) as an inhibitor of NF-κB activation by MSK1 by determining a decrease in the production of the proinflammatory cytokine in the presence of said test substance relative to the production of the proinflammatory cytokine in said cells in the absence of said test substance.

2. A method according to claim 1, wherein the proinflammatory cytokine is selected from the group consisting of TNF-α, IL-β, IL-6 and IL-8.

3. A method according to claim 2 wherein the proinflammatory cytokine is TNF-α.

4. A method according to claim 1, wherein, in step (a), MSK1 is contacted with the test substance or substances in the presence of a substrate comprising cyclic AMP response element binding protein (CREB) or activating transcription factor 1 (ATF-1) or fusion proteins thereof with glutathione S transferase or FLAG.

5. A method according to claim 1, wherein, in step (a), MSK1 is contacted with the test substance or substances in the presence of a substrate selected from the group consisting of the peptides
(SEQ ID NO:1)
Glu-Ile-Leu-Ser-Arg-Arg-Pro-Ser-Tyr-Arg-Lys,
(SEQ ID NO:2)
Gly-Arg-Pro-Arg-Thr-Ser-Ser-Phe-Ala-Glu-Gly,
(SEQ ID NO:3)
Lys-Lys-Arg-Asn-Arg-Thr-Leu-Ser-Val-Ala,
(SEQ ID NO:4)
Lys-Lys-Arg-Asn-Lys-Thr-Leu-Ser-Val-Ala and
(SEQ ID NO:5)
Lys-Lys-Leu-Asn-Arg-Thr-Leu-Ser-Val-Ala.

6. A method according to claim 1, wherein, in step (a), MSK1 is contacted with the test substance or substances in the presence of a substrate comprising an NF-κB subunit or a complex of NF-κB subunits, the subunits being selected from the group consisting of p50, p65 (Rel A), p52, c-rel and Rel B.

7. A method according to claim 1, wherein, in step (d), the test substance is identified as an inhibitor of NF-κB activation by MSK1 when MSK1 is inhibited by at least 10% in the presence of said test substance.

8. A method according to claim 7, wherein, in step (d), the test substance is identified as an inhibitor of NF-κB activation by MSK1 when MSK1 is inhibited by at least 25% in the presence of said test substance.

9. A method according to claim 1, wherein, in step (d), the test substance is identified as an inhibitor of NF-κB activation by MSK1 when MSK1 is inhibited by at least 50% in the presence of said test substance.

10. A method according to claim 1, wherein the MSK1 used in steps (a) and (b) is prepared by recombinant DNA techniques.

11. A method of identifying substances acting as inhibitors of the production of proinflammatory cytokines resulting from activation of mitogen and stress-activated protein kinase-1 (MSK1), the method comprising
(a) contacting MSK1, said MSK1 being in a form in which it is purified from other cellular proteins or other cellular components, with a test substance in the presence of a substrate for MSK1,
(b) measuring the level of phosphorylation by MSK1 of said substrate in the presence or absence of said test substance,
(c) identifying a test substance as an inhibitor of MSK1 when MSK1 activity is decreased in the presence of said test substance relative to the MSK1 activity in the absence of said test substance, and
(d) contacting said test substance with cells expressing activated MSK1 resulting in the production of proinflammatory cytokines and identifying a test substance identified as an inhibitor of MSK1 in step (c) as an inhibitor of the production of a proinflammatory cytokine resulting from activation of MSK1 by determining a decrease in the production of the proinflammatory cytokine in the presence of said test substance relative to the production of the proinflammatory cytokine in the absence of said test substance.

12. A method according to claim 11, wherein the proinflammatory cytokine is selected from the group consisting of TNF-α, IL-β IL-6 and IL-8.

13. A method according to claim 12, wherein the proinflammatory cytokine is TNF-α.

14. A method according to claim 11, wherein, in step (a), MSK1 is contacted with the test substance or substances in the presence of a substrate comprising cyclic AMP response element binding protein (CREB) or activating transcription factor 1 (ATF-1) or fusion proteins thereof with glutathione S transferase or FLAG.

15. A method according to claim 11, wherein, in step (a), MSK1 is contacted with the test substance or substances in the presence of a substrate selected from the group consisting of the peptides
(SEQ ID NO:1)
Glu-Ile-Leu-Ser-Arg-Arg-Pro-Ser-Tyr-Arg-Lys,
(SEQ ID NO:2)
Gly-Arg-Pro-Arg-Thr-Ser-Ser-Phe-Ala-Glu-Gly,
(SEQ ID NO:3)
Lys-Lys-Arg-Asn-Arg-Thr-Leu-Ser-Val-Ala,
(SEQ ID NO:4)
Lys-Lys-Arg-Asn-Lys-Thr-Leu-Ser-Val-Ala and
(SEQ ID NO:5)
Lys-Lys-Leu-Asn-Arg-Thr-Leu-Ser-Val-Ala.

16. A method according to claim 11, wherein, in step (a), MSK1 is contacted with the test substance or substances in the presence of a substrate comprising an NF-κB subunit or a complex of NF-κB subunits, the subunits being selected from the group consisting of p50, p65 (Rel A), p52, c-rel and Rel B.

17. A method according to claim 11, wherein, in step (d), the test substance is identified as an inhibitor of a proinflammatory cytokine produced as a result of activation of MSK1 when MSK1 is inhibited by at least 10% in the presence of said test substance.

18. A method according to claim 17, wherein, in step (d), the test substance is identified as an inhibitor of a proinflammatory cytokine produced as a result of activation of MSK1 when MSK1 is inhibited by at least 25% in the presence of said test substance.

19. A method according to claim 18, wherein, in step (d), the test substance is identified as an inhibitor of a proinflammatory cytokine produced as a result of activation of MSK1 when MSK1 is inhibited by at least 50% in the presence of said test substance.

20. A method according to claim 11, wherein the MSK1 used in steps (a) and (b) is prepared by recombinant DNA techniques.

21. A method of identifying substances acting as inhibitors of NF-κB activation by mitogen and stress-activated protein kinase-1 (MSK1), the method comprising
(a) contacting MSK1, said MSK1 being in a form in which it is purified from other cellular proteins or other cellular components, with a test substance in the presence of a substrate for MSK1,
(b) measuring the level of phosphorylation by MSK1 of said substrate in the presence or absence of said test substance,
(c) identifying a test substance as an inhibitor of MSK1 when MSK1 activity is decreased in the presence of said test substance relative to the MSK1 activity in the absence of said test substance, and
(d) contacting said test substance with cells transfected with an NF-κB-luciferase reporter plasmid, and identifying a test substance identified as an inhibitor of MSK1 in step (c) as an inhibitor of NF-κB activation by MSK1 by determining a decrease in the production of luciferase in the presence of said test substance relative to the production of luciferase in the absence of said test substance.

22. A method according to claim 21, wherein, in step (a), MSK1 is contacted with the test substance or substances in the presence of a substrate comprising cyclic AMP response element binding protein (CREB) or activating transcription factor 1 (ATF-1) or fusion proteins thereof with glutathione S transferase or FLAG.

23. A method according to claim 21, wherein, in step (a), MSK1 is contacted with the test substance or substances in the presence of a substrate selected from the group consisting of the peptides
(SEQ ID NO:1)
Glu-Ile-Leu-Ser-Arg-Arg-Pro-Ser-Tyr-Arg-Lys,
(SEQ ID NO:2)
Gly-Arg-Pro-Arg-Thr-Ser-Ser-Phe-Ala-Glu-Gly,
(SEQ ID NO:3)
Lys-Lys-Arg-Asn-Arg-Thr-Leu-Ser-Val-Ala,
(SEQ ID NO:4)
Lys-Lys-Arg-Asn-Lys-Thr-Leu-Ser-Val-Ala and
(SEQ ID NO:5)
Lys-Lys-Leu-Asn-Arg-Thr-Leu-Ser-Val-Ala.

24. A method according to claim 21, wherein, in step (a), MSK1 is contacted with the test substance or substances in the presence of a substrate comprising an NF-κB subunit or a complex of NF-κB subunits, the subunits being selected from the group consisting of p50, p65 (Rel A), p52, c-rel and Rel B.

25. A method according to claim 21, wherein, in step (d), the test substance is identified as an inhibitor of NF-κB activation by MSK1 when luciferase activity is inhibited by at least 10% in the presence of said test substance.

26. A method according to claim 21, wherein, in step (d), the test substance is identified as an inhibitor of NF-κB activation by MSK1 when luciferase activity is inhibited by at least 25% in the presence of said test substance.

27. A method according to claim 21, wherein, in step (d), the test substance is identified as an inhibitor of NF-κB activation by MSK1 when luciferase activity is inhibited by at least 50% in the presence of said test substance.

28. A method according to claim 21, wherein the MSK1 used in steps (a) and (b) is prepared by recombinant DNA techniques.

29. A method according to claim 21, wherein the cells transfected with the NF-κB-luciferase reporter plasmid in step (d) are cells naturally producing proinflammatory cytokines.

30. A method according to claim 29, wherein the cells are selected from the group consisting of leukocytes, peripheral blood mononuclear cells, monocytes, T-cells, macrophages, mast cells and endothelial cells.


SEQUENCE LISTING (20 sequences)

SEQ ID NO: 1 - protein, 11 aa, Artificial Sequence
  Synthetic Peptide Substrate for MSK1 or MSK2
EILSRRPSYRK

SEQ ID NO: 2 - protein, 11 aa, Artificial Sequence
  Synthetic Peptide Substrate for MSK1 or MSK2
GRPRTSSFAEG

SEQ ID NO: 3 - protein, 10 aa, Artificial Sequence
  Synthetic Peptide Substrate for MSK1 or MSK2
KKRNRTLSVA

SEQ ID NO: 4 - protein, 10 aa, Artificial Sequence
  Synthetic Peptide Substrate for MSK1 or MSK2
KKRNKTLSVA

SEQ ID NO: 5 - protein, 10 aa, Artificial Sequence
  Synthetic Peptide Substrate for MSK1 or MSK2
KKLNRTLSVA

SEQ ID NO: 6 - DNA, 21 bp, Artificial Sequence
  Synthetic NF-kappaB binding site
acagaggggactttccgagag

SEQ ID NO: 7 - DNA, 20 bp, Artificial Sequence
  Synthetic primer for N-terminal part of MSK1
tccgctgtctcctgggttcc

SEQ ID NO: 8 - DNA, 24 bp, Artificial Sequence
  Synthetic primer for N-terminal part of MSK1
gcactcctggcaacatttgtcact

SEQ ID NO: 9 - DNA, 22 bp, Artificial Sequence
  Synthetic primer for C-terminal part of MSK1
gtcagagggggagattcaggac

SEQ ID NO: 10 - DNA, 25 bp, Artificial Sequence
  Synthetic primer for C-terminal part of MSK1
atgagaccaacgggaaacattttta

SEQ ID NO: 11 - DNA, 66 bp, Artificial Sequence
  Synthetic primer to add FLAG epitope
gaggtaccgccaccatggactacaaggacgacgatgacaaggaggaggagggtggcagcagcggcg

SEQ ID NO: 12 - DNA, 25 bp, Artificial Sequence
  Synthetic primer to add FLAG epitope
cctgaaacagcttctcagaactctg

SEQ ID NO: 13 - DNA, 39 bp, Artificial Sequence
  Synthetic PCR primer with Nhe I site
acggctagcgatgtcccctatactaggttattggaaaat

SEQ ID NO: 14 - DNA, 23 bp, Artificial Sequence
  Oligonucleotide primer used for amplifying the GST and Multi Cloning Site from pGEX-4T-1
cagaggttttcaccgtcatcacc

SEQ ID NO: 15 - DNA, 29 bp, Artificial Sequence
  Synthetic PCR primer with EcoR I site
cgagaattcgaggaggagggtggcagcag

SEQ ID NO: 16 - DNA, 37 bp, Artificial Sequence
  Synthetic PCR primer with Not I site
gatgcggccgcctaagctactgagtccgagaactgga

SEQ ID NO: 17 - DNA, 23 bp, Artificial Sequence
  Synthetic PCR primer for spliced variant of CREB
gactctggagcagacaaccagca

SEQ ID NO: 18 - DNA, 25 bp, Artificial Sequence
  Synthetic PCR primer for spliced variant of CREB
atccagtccattttccaccacatag

SEQ ID NO: 19 - DNA, 25 bp, Artificial Sequence
  Synthetic PCR primer for MSK2
cgccatgggagacgaggatgaggac

SEQ ID NO: 20 - DNA, 21 bp, Artificial Sequence
  Synthetic PCR primer for MSK2
gcaccaggctcccggatcgga